(19) United States
(12) Patent Application Publication
(10) Pub. No.: US 2020/0390673 A1
(43) Pub. Date: Dec. 17, 2020

(54) COMPOSITION FOR NEUTRALIZING OR REDUCING EFFECTS OF SELF-DEFENSE SPRAYS

(71) Applicant: Christi Linford, Salt Lake City, UT (US)

(72) Inventor: Christi Linford, Salt Lake City, UT (US)

(21) Appl. No.: 16/800,740

(22) Filed: Feb. 25, 2020

Related U.S. Application Data
(60) Provisional application No. 62/860,401, filed on Jun. 12, 2019.

(51) Int. Cl. A61K 8/46; A61K 8/49; A61K 8/44; A61K 8/02; A61Q 19/10; A61K 8/34; A61K 8/24

(52) U.S. Cl. CPC A61K 8/463; A61K 8/4946; A61K 8/44; A61K 2800/87; A61Q 19/10; A61K 8/342; A61K 8/24; A61K 8/0208

(57) ABSTRACT

Disclosed herein are compositions for neutralizing the effects of self-defense sprays, and methods of using the same. In one embodiment, a composition comprises one or more anionic surfactants, an amphoteric surfactant, a viscosity agent, and an aqueous carrier.

### CROSS-REFERENCE TO RELATED APPLICATION(S)

This application claims the benefit of priority of U.S. Provisional Patent Application No. 62/860,401, filed Jun. 12, 2019, the disclosure of which is hereby incorporated by reference herein in its entirety.

### TECHNICAL FIELD

The present disclosure relates to compositions for removing irritants from a surface of the body. More particularly, the disclosure relates to compositions and formulations for neutralizing or reducing the effects of self-defense sprays, such as sprays containing oleoresin capsicum.

### BACKGROUND

Oleoresin capsicum is the active ingredient in pepper sprays used for personal self-defense or by law enforcement officers as a non-lethal means of subduing an individual. Once an individual is sprayed with pepper spray, the oleoresin capsicum oils cause severe burning and irritation of the skin and mucous membranes of the eyes, nose, throat, and lungs. This is the case, however, whether or not the person was intended to be incapacitated; if a police officer or security guard is accidentally sprayed, there is currently no remedy to quickly and efficiently alleviate the pain of the unintended recipient.

### SUMMARY

Disclosed herein are compositions for removing irritants from or neutralizing or reducing the effects of such irritants on a body surface when applied thereto, and methods of using the same. For example, such irritants may include oleoresin capsicum. In one aspect of the present disclosure, a composition comprises: one or more anionic surfactants from about 0.25% w/v to about 2.5% w/v; an amphoteric surfactant from about 2.0% w/v to about 4.0% w/v; a viscosity agent from about 0.5% w/v to about 1.5% w/v; an additional surfactant from about 0.01% w/v to about 0.2% w/v; and an aqueous carrier. Concentrations are calculated with respect to a total volume of the composition.

In one embodiment, the one or more anionic surfactants are present from about 0.75% w/v to about 2.0% w/v. In one embodiment, the one or more anionic surfactants comprise sodium laureth sulfate. In one embodiment, the sodium laureth sulfate is present from about 0.25% w/v to about 1.5% w/v. In one embodiment, the one or more one or more anionic surfactants further comprise one or more of sodium laureth-8 sulfate, magnesium laureth sulfate, magnesium laureth-8 sulfate, sodium oleth sulfate, or magnesium oleth sulfate.

In one embodiment, the additional surfactant comprises oleth-20.

In one embodiment, the composition further comprises one or more of a buffering agent, an anti-microbial agent, an anti-fungal agent, a preservative, a moisturizing agent, or a chelation agent.

In one embodiment, the composition is incorporated into a liquid, a gel, a paste, a cream, an emulsion, or a lotion. In one embodiment, the composition is incorporated into an absorbent matrix selected from a wipe, a tissue, a sponge, a towel, or a medical gauze.

In another aspect of the present disclosure, a composition comprises effective amounts of sodium laureth sulfate, disodium cocoamphodiacetate, hexylene glycol, and an aqueous carrier for neutralizing or reducing effects of an irritant on a body surface when applied thereto.

In one embodiment, the composition further comprises one or more of one or more of sodium laureth-8 sulfate, magnesium laureth sulfate, magnesium laureth-8 sulfate, sodium oleth sulfate, or magnesium oleth sulfate.

In one embodiment, the composition further comprises oleth-20.

In one embodiment, the composition further comprises dipotassium phosphate.

In one embodiment, the composition further comprises allantoin.

In one embodiment, the composition further comprises disodium ethylenediaminetetraacetic (EDTA).

In one embodiment, the composition further comprises one or more of imidazolidinyl urea, methylparaben, or sodium benzoate.

In one embodiment, the composition further comprises the sodium laureth sulfate is present from about 0.25% w/v to about 1.5% w/v, calculated with respect to a total volume of the composition.

In another aspect of the present disclosure, a method of treating a body surface that has been exposed to oleoresin capsicum comprises applying to the body surface an effective amount of an aqueous composition comprising an anionic surfactant mixture. In some embodiments, the anionic surfactant mixture comprises sodium laureth sulfate.

In one embodiment, the anionic surfactant mixture further comprises one or more of sodium laureth-8 sulfate, magnesium laureth sulfate, magnesium laureth-8 sulfate, sodium oleth sulfate, or magnesium oleth sulfate.

In one embodiment, the aqueous composition is incorporated into a liquid, a gel, a paste, a cream, an emulsion, or a lotion. In on embodiment, the aqueous composition is incorporated into an absorbent matrix selected from a group consisting of a wipe, a tissue, a sponge, a towel, and a medical gauze.

Other aspects of the present disclosure include liquids, gels, pastes, creams, emulsions, or lotions incorporating any of the aforementioned compositions, as well as absorbent matrices, such as wipes, tissues, sponges, towels, or medical gauzes, incorporating any of the aforementioned compositions.

As used herein, the term “viscosity agent” refers to any compound or mixture of compounds that increase the viscosity of a liquid while having a negligible or substantially low impact on other properties of the liquid.

Also as used herein, the term “foam boosting agent” refers to any compound or mixture of compounds that facilitates the formation of foam (such as an amphoteric surfactant).

Also as used herein, the term “buffering agent” refers to a weak acid or a weak base used to buffer the pH of a solution and prevent rapid changes in pH when acidic or basic species are introduced.

Also as used herein, the term “moisturizing agent” refers to any compound or mixture of compounds that, when applied to human skin, help to improve and maintain skin moisture.

Also as used herein, the term “chelation agent” refers to a compound or mixture of compounds that bind to metals, minerals, or toxins such that the effects of such metals, minerals, or toxins when bound are suppressed.

Also as used herein, the term “effective amount” refers to the amount of a component, either alone or in combination with others, sufficient to confer a therapeutic effect when applied to skin or mucous membranes of a subject that have been exposed to an irritant (such as oleoresin capsicum) such that the irritant is removed or the effects of the irritant are neutralized or reduced faster than the application of a similar composition that either lacks the component or includes an insufficient amount of the component.

Also as used herein, the term “treating” refers to the application of a composition to skin or mucous membranes of a subject that have been exposed to an irritant (such as oleoresin capsicum) with the purpose of removing the irritant from or neutralizing or reducing the effects of the irritant on the skin or mucous membranes.

Also as used herein, all occurrences of “% w/v” refer to a weight to volume percentage as a ratio of grams per 100 milliliters. For example, if a 100 milliliter (mL) composition contains 10 grams (g) of component A, then component A is present at 10% w/v of the composition.

Also as used herein, all occurrences of “% w/w” refer to a weight to weight percentage. For example, if a 100 g composition contains 10 g of component A, then component A is present at 10% w/w of the composition.

Also as used herein, the term “about,” when used in connection with a measurable quantity, refers to the normal variations in that measurable quantity, as expected by one of ordinary skill in the art in making the measurement and exercising a level of care commensurate with the objective of the measurement and the precision of the measuring equipment. In certain embodiments, the term “about” includes the recited number ±10%, such that “about 10” would include from 9 to 11.

Other terms used herein not explicitly defined above should be construed in accordance with their broadest reasonable meaning as would be understood by one of ordinary skill in the art who is familiar with such terminology.

### DETAILED DESCRIPTION

Certain embodiments of the present disclosure relate to a cleansing composition for removing oleoresin capsicum from or neutralizing or reducing the effects of oleoresin capsicum on body surfaces (e.g., eyes, the nose, the mouth, etc.) that have been contacted with oleoresin capsicum (e.g., upon being sprayed by a pepper spray composition). The embodiments of the present disclosure allow for fast and effective removal of oleoresin capsicum oils from the body surfaces to quickly alleviate the pain without the need to continuously flush the affected area of skin with water.

The composition can be applied to an individual's face to remove the oleoresin capsicum oil and reduce the inflammation and burning. In some embodiments, the composition is stored in a plastic bottle having a dispensing top or a spray nozzle. The composition can be applied to a soft material such as a paper towel, and then applied to the affected area. Without being bound by theory, it is believed the various surfactants included in the composition coat and bind to the capsicum oils, which facilitates removal from the surface of the skin or membranes of the individual being treated.

In some embodiments, an exemplary composition comprises the following components: deionized water, disodium cocoamphodiacetate, hexylene glycol, sodium laureth sulfate, sodium laureth-8 sulfate, magnesium laureth sulfate, magnesium laureth-8 Sulfate, sodium oleth Sulfate, magnesium oleth sulfate, dipotassium phosphate, imidazolidinyl urea, allantoin, methylparaben, oleth 20, disodium EDTA, and sodium benzoate.

In some embodiments, an exemplary composition comprises one or more anionic surfactants, an amphoteric surfactant, a viscosity agent, an additional surfactant, and an aqueous carrier (e.g., deionized water).

In some embodiments, the one or more anionic surfactants are present (e.g., as a mixture of surfactants) such that a total amount of anionic surfactants ranges from about 0.05% w/v to about 4% w/v, from 0.15% w/v to about 3.5% w/v, from about 0.25% w/v to about 2.5% w/v, or from about 0.75% w/v to about 2.0% w/v. In some embodiments, the one or more anionic surfactants are present at about 0.05% w/v, about 0.10% w/v, about 0.15% w/v, about 0.20% w/v, about 0.25% w/v, about 0.30% w/v, about 0.35% w/v, about 0.40% w/v, about 0.45% w/v, about 0.50% w/v, about 0.55% w/v, about 0.60% w/v, about 0.65% w/v, about 0.70% w/v, about 0.75% w/v, about 0.80% w/v, about 0.85% w/v, about 0.90% w/v, about 0.95% w/v, about 1.0% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2.0% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3.0% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4.0% w/v, or within a range defined by any combination thereof (e.g., about 0.75% w/v to about 1.5% w/v, about 1.0% w/v to about 3.5% w/v, etc.). In some embodiments, sodium laureth sulfate may be present, by itself or in combination with other anionic surfactants, in any of the aforementioned ranges. For example, a total amount of sodium laureth sulfate may be present in the composition from about 0.25% w/v to about 1.5% w/v.

In some embodiments, the one or more anionic surfactants may include sodium laureth sulfate, sodium laureth-8 sulfate, magnesium laureth sulfate, magnesium laureth-8 sulfate, sodium oleth sulfate, magnesium oleth sulfate, another suitable anionic surfactant, or combinations thereof in any of the aforementioned ranges. For example, the composition may be prepared by adding about 2.0% w/v to about 3.0% w/v of Texapon® ASV 50, which is a surfactant mixture for use in shampoos, skin cleansers, and bath, and shower products (available from BASF). The composition of Texapon® ASV 50 is shown in Table 2.

TABLE 1
Composition of Texapon ® ASV 50

| Component | Amount (% w/w) |
|---|---|
| Water | >45-55 |
| Sodium laureth sulfate | >15-25 |
| Sodium laureth-8 sulfate | >15-25 |
| Magnesium laureth sulfate | >2-7 |
| Magnesium laureth-8 sulfate | >2-7 |
| Sodium oleth sulfate | >2-7 |
| Magnesium oleth sulfate | <2 |

In some embodiments, the amphoteric surfactant is present (e.g., as a single type of amphoteric surfactant or as a mixture of amphoteric surfactants) such that a total amount of amphoteric surfactant ranges from about 0.5% w/v to about 6% w/v, from 1.0% w/v to about 5.0% w/v, or from about 2.0% w/v to about 4.0% w/v. In some embodiments, the amphoteric surfactant is present at about 1.0% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2.0% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3.0% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4.0% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5.0% w/v, about 5.1% w/v, about 5.2% w/v, about 5.3% w/v, about 5.4% w/v, about 5.5% w/v, about 5.6% w/v, about 5.7% w/v, about 5.8% w/v, about 5.9% w/v, about 6.0% w/v, or within a range defined by any combination thereof (e.g., about 2.0% w/v to about 4.0% w/v, about 1.5% w/v to about 2.5% w/v, etc.). In some embodiments, the amphoteric surfactant comprises disodium cocoamphodiacetate.

In some embodiments, the viscosity agent is present (e.g., as a single type of viscosity agent or as a mixture of viscosity agents) such that a total amount of viscosity agent ranges from about 0.1% w/v to about 4% w/v, from 0.25% w/v to about 2.0% w/v, or from about 0.5% w/v to about 1.5% w/v. In some embodiments, the viscosity agent is present at about 0.05% w/v, about 0.10% w/v, about 0.15% w/v, about 0.20% w/v, about 0.25% w/v, about 0.30% w/v, about 0.35% w/v, about 0.40% w/v, about 0.45% w/v, about 0.50% w/v, about 0.55% w/v, about 0.60% w/v, about 0.65% w/v, about 0.70% w/v, about 0.75% w/v, about 0.80% w/v, about 0.85% w/v, about 0.90% w/v, about 0.95% w/v, about 1.0% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2.0% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3.0% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4.0% w/v, or within a range defined by any combination thereof (e.g., about 0.75% w/v to about 1.5% w/v, about 1.0% w/v to about 3.5% w/v, etc.). In some embodiments, the viscosity agent comprises hexylene glycol.

In some embodiments, the additional surfactant is present (e.g., as a single type of surfactant or as a mixture of surfactants) such that a total amount of additional surfactant ranges from about 0.01% w/v to about 1% w/v, from 0.01% w/v to about 0.5% w/v, or from about 0.01% w/v to about 0.2% w/v. In some embodiments, the viscosity agent is present at about 0.01% w/v, about 0.02% w/v, about 0.03% w/v, about 0.04% w/v, about 0.05% w/v, about 0.06% w/v, about 0.07% w/v, about 0.08% w/v, about 0.09% w/v, about 0.10% w/v, about 0.15% w/v, about 0.20% w/v, about 0.25% w/v, about 0.30% w/v, about 0.35% w/v, about 0.40% w/v, about 0.45% w/v, about 0.50% w/v, about 0.55% w/v, about 0.60% w/v, about 0.65% w/v, about 0.70% w/v, about 0.75% w/v, about 0.80% w/v, about 0.85% w/v, about 0.90% w/v, about 0.95% w/v, about 1.0% w/v, or within a range defined by any combination thereof (e.g., about 0.01% w/v to about 0.2% w/v, about 0.05% w/v to about 0.5% w/v, etc.).

In some embodiments, the additional surfactant may be a type of surfactant other than an anionic surfactant (e.g., an ionic surfactant). In some embodiments, the additional surfactant may be any surfactant other than sodium laureth sulfate, sodium laureth-8 sulfate, magnesium laureth sulfate, magnesium laureth-8 sulfate, sodium oleth sulfate, or magnesium oleth sulfate. In some embodiments, the additional surfactant is a polyethylene glycol ether of oleyl alcohol, such as, for example, oleth-2, oleth-3, oleth-4, oleth-5, oleth-6, oleth-7, oleth-8, oleth-9, oleth-10, oleth-11, oleth-12, oleth-15, oleth-16, oleth-20, oleth-23, oleth-25, oleth-30, oleth-40, oleth-44, oleth-50, or combinations thereof. In some embodiments, the additional surfactant is oleth-20.

In some embodiments, the composition further comprises a buffering agent, an anti-microbial agent, an anti-fungal agent, a preservative, a moisturizing agent, a chelation agent, or a combination thereof.

In some embodiments, the buffering agent is present from about 0.1% w/v to about 0.5% w/v, about 0.15% w/v to about 0.45% w/v, or about 0.2% w/v to about 0.4% w/v. In some embodiments, the buffering agent comprises a phosphate (e.g., dipotassium phosphate), which results in a phosphate buffer being present in the composition.

In some embodiments, the anti-microbial agent is present from about 0.1% w/v to about 0.5% w/v, about 0.15% w/v to about 0.45% w/v, or about 0.2% w/v to about 0.4% w/v. In some embodiments, the anti-microbial agent comprises imidazolidinyl urea.

In some embodiments, the anti-fungal agent is present from about 0.05% w/v to about 0.4% w/v, about 0.1% w/v to about 0.35% w/v, or about 0.15% w/v to about 0.3% w/v. In some embodiments, the anti-fungal agent comprises methylparaben.

In some embodiments, the preservative is present from about 0.01% w/v to about 0.05% w/v, or about 0.02% w/v to about 0.04% w/v. In some embodiments, the preservative comprises sodium benzoate.

In some embodiments, the moisturizing agent is present from about 0.05% w/v to about 0.4% w/v, about 0.1% w/v to about 0.35% w/v, or about 0.15% w/v to about 0.3% w/v. In some embodiments, the moisturizing agent comprises a diureide of glyoxylic acid (e.g., allantoin).

In some embodiments, the chelation agent is present from about 0.02% w/v to about 0.10% w/v, or about 0.04% w/v to about 0.06% w/v. In some embodiments, the chelation agent comprises one or more of disodium EDTA, nitrilotris(methylene)triphosphonic acid solution, sodium triphosphate pentabasic, N-carboxymethylated polyethyleneimine, or 1-hydroxyethane 1,1-diphosphonic acid or salts thereof.

In some embodiments, the composition is formulated such that water accounts for most of the weight of the composition (e.g., greater than or equal to 50% w/v, greater than or equal to 60% w/v, greater than or equal to 70% w/v, greater than or equal to 80% w/v, or greater than or equal to 90% w/v). Such compositions may be packaged and shipped, and may be used directly from the package without dilution by water or any additional components. In some embodiments, the composition is formulated as a concentrated solution (e.g., less than 50% w/v). Such compositions may be packaged and shipped, but should be diluted, for example, with deionized water prior to use to ensure effective treatment.

In some embodiments, the composition is incorporated into a liquid, a gel, a paste, a cream, an emulsion, or a lotion. The additional components to produce such formulations are within the purview of one of ordinary skill in the art. In some embodiments, the composition is incorporated into an absorbent matrix, such as a wipe, a tissue, a sponge, a towel, and a medical gauze. In some embodiments, a kit may comprise a container that holds the composition (e.g., a plastic body), as well as wipes, tissues, or other materials that may facilitate application of the composition to a body surface.

In some embodiments, a method of treating a body surface that has been exposed to oleoresin capsicum comprises applying to the body surface an effective amount of any of the compositions described herein. For example, to treat an individual's face that has been exposed to oleoresin capsicum, one or more volumes of the composition may be applied directly to the individual's face. For example, one or more volumes of 10 mL to 100 mL may be applied in succession to the individual's face until the pain sensation subsides or is reduced to a tolerable level.

### Illustrative Examples

The following examples are set forth to assist in understanding the embodiments described herein and should not be construed as specifically limiting the embodiments described and claimed herein. Such variations, including the substitution of all equivalents now known or later developed, which would be within the purview of those skilled in the art, and changes in formulation or minor changes in experimental design, are to be considered to fall within the scope of the embodiments incorporated herein.

### Example 1

A composition was prepared by adding the following components to 91 grams of deionized water: disodium cocoamphodiacetate (liquid), Texapon® ASV 50 (liquid), hexylene glycol (liquid), sodium laureth sulfate (liquid), dipotassium phosphate (dry powder), imidazolidinyl urea (dry powder), allantoin (dry powder), methylparaben (dry powder), oleth 20 (liquid), disodium EDTA (dry powder), and sodium benzoate (dry powder). The components were added in quantities summarized in Table 2 below, and mixed at room temperature (70-80° F.) without heating.

TABLE 2
Exemplary Composition

| | Amount | |
|---|---|---|
| Component | (% w/v) | Description/Function |
| Deionized water | 91.00 | Carrier |
| Disodium | 3.58 | Foam boosting agent; synthetic |
| cocoamphodiacetate | | amphoteric surfactant |
| Texapon ® ASV 50 | 2.46 | Surfactant mixture |
| Hexylene glycol | 1.25 | Viscosity agent |
| Sodium laureth | 0.49 | Anionic surfactant |
| sulfate (50%) | | |
| Dipotassium | 0.31 | Buffering agent |
| phosphate | | |
| Imidazolidinyl urea | 0.30 | Anti-microbial agent |
| Allantoin | 0.20 | Moisturizing agent; |
| | | diureide of glyoxylic acid |
| Methylparaben | 0.20 | Anti-fungal agent |
| Oleth 20 | 0.13 | Surfactant; polyethylene |
| | | glycol ether of oleyl alcohol |
| Disodium EDTA | 0.05 | Chelation agent |
| Sodium benzoate | 0.03 | Preservative |

### Example 2

A composition was prepared similar to Example 1, except that sodium laureth sulfate was not added and was instead replaced with additional deionized water. Thus, the sodium laureth sulfate present in this composition was provided the Texapon® ASV 50 without any additional added sodium laureth sulfate. Compositions containing lower amounts of anionic surfactant may be utilized to reduce potential skin and/or eye irritation.

The use of the terms “a,” “an,” “the,” and similar referents in the context of describing the materials and methods discussed herein (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the materials and methods and does not pose a limitation on the scope unless otherwise claimed. No language in the specification should be construed as indicating any non-claimed element as essential to the practice of the disclosed materials and methods.

Reference throughout this specification to “one embodiment,” “certain embodiments,” “one or more embodiments” or “an embodiment” means that a particular feature, structure, material, or characteristic described in connection with the embodiment is included in at least one embodiment of the present disclosure. Thus, the appearances of the phrases such as “in one or more embodiments,” “in certain embodiments,” “in one embodiment,” or “in an embodiment” in various places throughout this specification are not necessarily referring to the same embodiment of the present disclosure. Furthermore, the particular features, structures, materials, or characteristics may be combined in any suitable manner in one or more embodiments.

Although the embodiments disclosed herein have been described with reference to particular embodiments, it is to be understood that these embodiments are merely illustrative of the principles and applications of the present disclosure. It will be apparent to those skilled in the art that various modifications and variations can be made to the compositions of the present disclosure without departing from the spirit and scope of the disclosure. Thus, it is intended that the present disclosure include modifications and variations that are within the scope of the appended claims and their equivalents, and the above-described embodiments are presented for purposes of illustration and not of limitation.

What is claimed is:

1. A composition to remove irritants from a body surface when applied thereto, the composition comprising:
one or more anionic surfactants from about 0.25% w/v to about 2.5% w/v;
an amphoteric surfactant from about 2.0% w/v to about 4.0% w/v;
a viscosity agent from about 0.5% w/v to about 1.5% w/v;
an additional surfactant from about 0.01% w/v to about 0.2% w/v; and
an aqueous carrier,
wherein concentrations are calculated with respect to a total volume of the composition.

2. The composition of claim 1, wherein the one or more anionic surfactants are present from about 0.75% w/v to about 2.0% w/v.

3. The composition of claim 1, wherein the one or more anionic surfactants comprise sodium laureth sulfate, and wherein the sodium laureth sulfate is present from about 0.25% w/v to about 1.5% w/v.

4. The composition of claim 1, wherein the one or more anionic surfactants further comprise one or more of sodium laureth-8 sulfate, magnesium laureth sulfate, magnesium laureth-8 sulfate, sodium oleth sulfate, or magnesium oleth sulfate.

5. The composition of claim 1, wherein the additional surfactant comprises oleth-20.

6. The composition of claim 1, wherein the composition further comprises one or more of a buffering agent, an anti-microbial agent, an anti-fungal agent, a preservative, a moisturizing agent, or a chelation agent.

7. The composition of claim 1, wherein the composition is incorporated into a liquid, a gel, a paste, a cream, an emulsion, or a lotion.

8. The composition of claim 1, wherein the composition is incorporated into an absorbent matrix selected from a group consisting of a wipe, a tissue, a sponge, a towel, and a medical gauze.

9. A composition for neutralizing or reducing effects of an irritant on a body surface when applied thereto, the composition comprising effective amounts of:
sodium laureth sulfate;
disodium cocoamphodiacetate;
hexylene glycol; and
an aqueous carrier.

10. The composition of claim 9, further comprising one or more of one or more of sodium laureth-8 sulfate, magnesium laureth sulfate, magnesium laureth-8 sulfate, sodium oleth sulfate, or magnesium oleth sulfate.

11. The composition of claim 9, further comprising oleth-20.

12. The composition of claim 9, further comprising dipotassium phosphate.

13. The composition of claim 9, further comprising allantoin.

14. The composition of claim 9, further comprising disodium EDTA.

15. The composition of claim 9, further comprising one or more of imidazolidinyl urea, methylparaben, or sodium benzoate.

16. The composition of claim 9, wherein the sodium laureth sulfate is present from about 0.25% w/v to about 1.5% w/v, calculated with respect to a total volume of the composition.

17. A method of treating a body surface that has been exposed to oleoresin capsicum, comprising applying to the body surface an effective amount of an aqueous composition comprising an anionic surfactant mixture, the anionic surfactant mixture comprising sodium laureth sulfate.

18. The method of claim 17, wherein the anionic surfactant mixture further comprises one or more of sodium laureth-8 sulfate, magnesium laureth sulfate, magnesium laureth-8 sulfate, sodium oleth sulfate, or magnesium oleth sulfate.

19. The method of claim 17, wherein the aqueous composition is incorporated into a liquid, a gel, a paste, a cream, an emulsion, or a lotion.

20. The method of claim 17, wherein the aqueous composition is incorporated into an absorbent matrix selected from a group consisting of a wipe, a tissue, a sponge, a towel, and a medical gauze.
